(19) United States
(12) Patent Application Publication
(10) Pub. No.: US 2022/0364038 A1
(43) Pub. Date: Nov. 17, 2022

(54) SUBSTANCE INTRODUCTION DEVICE AND SUBSTANCE INTRODUCTION METHOD

(71) Applicant: TERUMO KABUSHIKI KAISHA, Tokyo (JP)

(72) Inventors: YU NAKANO, Kanagawa (JP); YUSUKE NOZAKI, Kanagawa (JP)

(21) Appl. No.: 17/879,498

(22) Filed: Aug. 2, 2022

Related U.S. Application Data
(63) Continuation of application No. PCT/JP2021/007526, filed on Feb. 26, 2021.

(30) Foreign Application Priority Data
Mar. 6, 2020 (JP) 2020-039338

(51) Int. Cl. C12M 1/42; C12M 1/00; C12M 3/00; C12N 15/87

(52) U.S. Cl. CPC C12M 35/02; C12M 23/40; C12M 23/44; C12N 15/87

(57) ABSTRACT

A substance introduction device according to the present disclosure is a substance introduction device that introduces the substance into the cell by the electroporation, the substance introduction device including: a storage unit that stores the cell suspension containing the cells and the substance; an introduction path that introduces the cell suspension into the storage unit; a filling unit that fills the storage unit with the predetermined amount of the cell suspension from the introduction path; an electrode pair that applies a voltage to the cell suspension stored in the storage unit; and a discharge path that discharges the cell suspension after the voltage is applied from the storage unit.

### CROSS REFERENCE TO RELATED APPLICATIONS

The present application is a continuation of and claims benefit to PCT Application No. PCT/JP2021/007526 filed on Feb. 26, 2021, entitled “SUBSTANCE INTRODUCTION DEVICE AND SUBSTANCE INTRODUCTION METHOD” which claims priority to Japanese Patent Application No. 2020-039338 filed on Mar. 6, 2020. The entire disclosure of the applications listed above are hereby incorporated herein by reference, in their entirety, for all that they teach and for all purposes.

### BACKGROUND

The present disclosure relates to a substance introduction device and a substance introduction method.

Conventionally, a technique for acquiring a large amount of cells into which a substance, such as DNA, is introduced by electroporation is known. For example, Japanese Patent No. 3740664 generally discloses a liquid circulation device that continuously performs electroporation by circulating a cell suspension containing cells and a substance to be introduced into the cells.

### SUMMARY

However, in the electroporation, optimum conditions for performing electroporation such as a voltage value of voltage to be applied or an applied time are different depending on environmental factors such as an amount of the cell suspension to be processed at one time, a volume of a container in which the process is performed, and a distance between an electrode pair to which a voltage is applied. Therefore, for example, even in a case where the conditions for performing the electroporation for a small amount of cells is established in a certain container, when the electroporation is performed in a different container in order to increase the amount of cell suspension to be processed at one time, it is necessary to reconfigure the conditions in the different container. Therefore, there is a demand for improvement of a usefulness of a technique for introducing a substance into cells by the electroporation by making it possible to change a total amount of the cell suspension to be electroporated under the same conditions for performing.

An object of the present disclosure made in view of such circumstances is to provide a substance introduction device and a substance introduction method that improves the usefulness of the technique for introducing a substance into a cell by the electroporation.

A substance introduction device according to at least one embodiment of the present disclosure is a substance introduction device that introduces a substance into at least one cell by electroporation, the substance introduction device including: a storage unit that stores a cell suspension containing the at least one cell and the substance; an introduction path that introduces the cell suspension into the storage unit; a filling unit that fills the storage unit with a predetermined amount of the cell suspension from the introduction path; an electrode pair that applies a voltage to the cell suspension stored in the storage unit; and a discharge path that discharges the cell suspension from the storage unit after the voltage is applied.

In some embodiments of the present disclosure, the substance introduction device further includes a control unit that controls the filling unit to fill the predetermined amount of the cell suspension in the storage unit.

In some embodiments of the present disclosure, the substance introduction device further includes a first opening/closing unit that selectively opens and closes the introduction path and a second opening/closing unit that selectively opens and closes the discharge path, and the control unit controls opening and closing of at least one of the first opening/closing unit and the second opening/closing unit, and controls at least one of introduction of the cell suspension into the storage unit and/or discharge of the cell suspension.

In some embodiments of the present disclosure, the substance introduction device further includes a liquid amount measurement unit that measures a liquid amount of the cell suspension stored in the storage unit, and the control unit controls the filling unit to fill the storage unit with the cell suspension so that the liquid amount measured by the liquid amount measurement unit falls within a predetermined range.

In some embodiments of the present disclosure, the substance introduction device further includes a resistance measurement unit (e.g., electrical sensor, etc.) that measures a resistance value between the electrode pair, and the control unit controls the filling unit to adjust the predetermined amount of the cell suspension stored in the storage unit so that the resistance value measured by the resistance measurement unit falls within a predetermined range.

In some embodiments of the present disclosure, the substance introduction device further includes an auxiliary introduction path that introduces a liquid different from the cell suspension into the storage unit, and the filling unit fills the storage unit with a predetermined amount of the liquid from the auxiliary introduction path.

In some embodiments of the present disclosure, in the substance introduction device, an introduction port through which the introduction path communicates with the storage unit is provided at a position different from a position of a discharge port through which the discharge path communicates with the storage unit.

In some embodiments of the present disclosure, in the substance introduction device, the storage unit is detachable from the substance introduction device.

A substance introduction method according to at least one embodiment of the present disclosure is the substance introduction method executed by a substance introduction device that introduces a substance into at least one cell by electroporation, the method including: providing the substance introduction device, wherein the substance introduction device includes a storage unit that stores a cell suspension containing the at least one cell and the substance; an introduction path that introduces the cell suspension into the storage unit; a filling unit that fills the storage unit with a predetermined amount of the cell suspension from the introduction path; an electrode pair that applies a voltage to the cell suspension stored in the storage unit; and a discharge path that discharges the cell suspension from the storage unit after the voltage is applied; filling the storage unit with the predetermined amount of the cell suspension; and applying the voltage to the cell suspension stored in the storage unit.

A substance introduction device according to at least one embodiment of the present disclosure is a substance introduction device that introduces a substance into a cell by electroporation, the substance introduction device including: a storage unit that stores a cell suspension containing the cell and the substance; an introduction path that introduces the cell suspension into the storage unit; an electrode pair that applies a voltage to the cell suspension stored in the storage unit; a discharge path that discharges the cell suspension from the storage unit after the voltage is applied; a first opening/closing unit that opens and closes the introduction path; a second opening/closing unit that opens and closes the discharge path; and a control unit that controls opening and closing of at least one of the first opening/closing unit and the second opening/closing unit, and controls introduction of the cell suspension into the storage unit or discharge of the cell suspension.

With the substance introduction device and the substance introduction method according to embodiments of the present disclosure, the usefulness of the technique for introducing a substance into cells by electroporation can be improved when compared to conventional devices and methods.

### BRIEF DESCRIPTION OF THE DRAWINGS

FIG. 1 is a schematic diagram illustrating a schematic configuration of a substance introduction device according to a first embodiment of the present disclosure using a partial cross-sectional view;

FIG. 2 is a flowchart illustrating an operation of the substance introduction device according to the first embodiment of the present disclosure;

FIG. 3 is a schematic diagram illustrating a schematic configuration of a substance introduction device according to a second embodiment of the present disclosure;

FIG. 4 is a flowchart illustrating an operation of the substance introduction device according to the second embodiment of the present disclosure; and

FIG. 5 is a schematic diagram illustrating a schematic configuration of a substance introduction device according to a third embodiment of the present disclosure.

### DETAILED DESCRIPTION

Hereinafter, a substance introduction device 1 according to embodiments of the present disclosure will be described with reference to the drawings.

In the drawings, the same or corresponding parts are denoted by the same reference numerals. In the description of the present embodiment, the description of the same or corresponding parts will be omitted or simplified as appropriate.

The substance introduction device 1, according to embodiments of the present disclosure, introduces a substance into a cell by electroporation. The electroporation is a method of making a hole or an opening in a cell membrane of a cell and introducing the substance into the cell by applying an electric pulse or the like to the cell and the cell suspension containing a substance to be introduced into the cell. In the present embodiment, the cell suspension is obtained by suspending, for example, an animal cell or a cell such as Escherichia coli and a substance such as a gene or a protein in a buffer fluid (e.g., buffer).

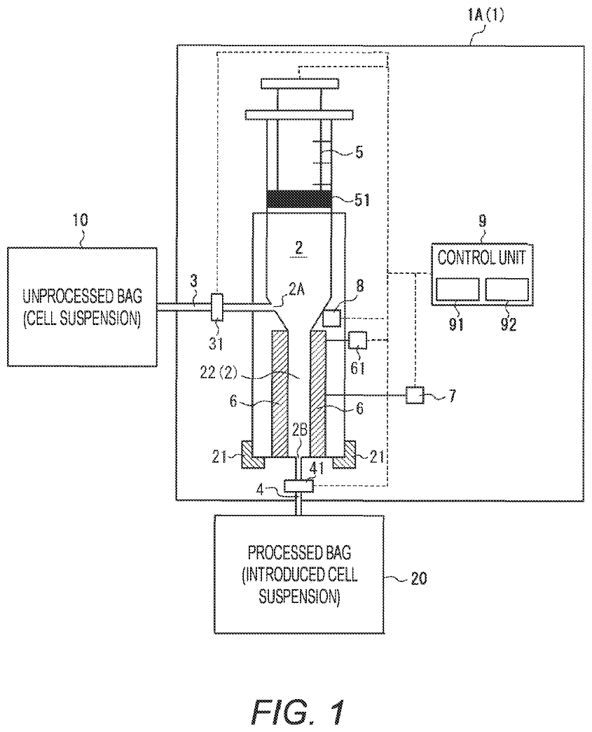

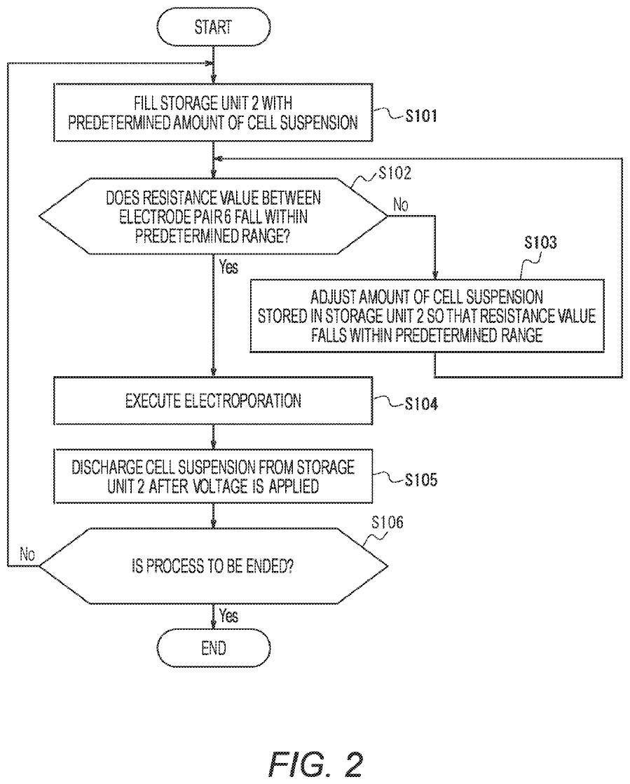

A substance introduction device 1A, which is the first embodiment of the substance introduction device 1 according to the present disclosure, will be described with reference to FIGS. 1 and 2. FIG. 1 is a schematic diagram illustrating a schematic configuration of the substance introduction device 1A according to the first embodiment of the present disclosure using a partial cross-sectional view. As illustrated in FIG. 1, the substance introduction device 1A includes a storage unit 2 that stores the cell suspension, an introduction path 3 that introduces the cell suspension into the storage unit 2, a filling unit 5 that fills the storage unit 2 with the predetermined amount of the cell suspension from the introduction path 3, an electrode pair 6 that applies a voltage to the cell suspension stored in the storage unit 2, and a discharge path 4 that discharges the cell suspension after the voltage is applied from the storage unit 2. An unprocessed bag 10 that stores a cell suspension before the electroporation is performed is connected to the introduction path 3. A processed bag 20 that stores a cell suspension after the electroporation is performed is connected to the discharge path 4.

As a result, in the substance introduction device 1A, when filling is performed by the filling unit 5, the predetermined amount of the cell suspension is stored in the storage unit 2 from the unprocessed bag 10 through the introduction path 3. The electroporation is performed by applying a voltage to the cell suspension stored in the storage unit 2 by the electrode pair 6. After the electroporation is performed, the cell suspension stored in the storage unit 2, which is after the voltage is applied, is discharged to the processed bag 20 through the discharge path 4. After discharging the cell suspension, the substance introduction device 1A refills the storage unit 2 with the predetermined amount of the cell suspension from the unprocessed bag 10 by the filling unit 5, and performs the electroporation. As described above, by repeatedly performing the electroporation for each predetermined amount of cell suspension, the electroporation can be repeated under the same conditions for performing and the usefulness of the technique for introducing the substance into cells by the electroporation can be improved even when a total amount of the cell suspension to be electroporated is changed.

Next, the configuration of the substance introduction device 1A will be described in detail with reference to FIG. 1. As illustrated in FIG. 1, the substance introduction device 1A includes the storage unit 2, the introduction path 3, the discharge path 4, the filling unit 5, the electrode pair 6, a resistance measurement unit 7, a liquid amount measurement unit 8, and a control unit 9.

The storage unit 2 is configured to store the cell suspension containing cells and the substance to be introduced into the cells. The storage unit 2 has an elongated space therein. A size of the space inside the storage unit 2 is determined according to the amount of the cell suspension to be processed in one electroporation, and is, for example, 10 microliters to 10 milliliters, and desirably 40 microliters to 400 microliters. In the present embodiment, the storage unit 2 is a cuvette as an example of the container detachable from the substance introduction device 1A. More specifically, the substance introduction device 1A of the present embodiment includes a holding unit 21 that detachably holds the container as the storage unit 2. When the container serving as the storage unit 2 is attached to the holding unit 21, an internal space of the container communicates with the introduction path 3 and the discharge path 4. However, the storage unit 2 may be formed integrally with the substance introduction device 1A and is not necessarily detachable from the substance introduction device 1A.

The storage unit 2 is provided with an introduction port 2A communicating with the introduction path 3 and a discharge port 2B communicating with the discharge path 4. The introduction port 2A is provided on a side surface located around a peripheral orthogonal to the longitudinal direction of the storage unit 2. In some examples, an axis of the introduction port 2A and/or the introduction path 3 may be arranged orthogonally to a longitudinal axis of the storage unit and/or the discharge port. The discharge port 2B is provided at one end in the longitudinal direction of the storage unit 2. That is, the introduction port 2A through which the introduction path 3 communicates with the storage unit 2 is provided at a position different from the discharge port 2B through which the discharge path 4 communicates with the storage unit 2. The filling unit 5 is connected to one end different from the one end provided with the discharge port 2B in the longitudinal direction of the storage unit 2. The storage unit 2 may be formed from, for example, glass, plastic, silicon, quartz, and/or the like.

The introduction path 3 is configured to introduce the cell suspension into the storage unit 2. In the present embodiment, the introduction path 3 is made with a tubular member. The introduction path 3 may be formed integrally with the storage unit 2 in advance or may be attached by a valve, a connector, or the like so that a liquid such as the cell suspension does not leak to the outside from a connection part with the storage unit 2. The introduction path 3 may be formed from the same material as the storage unit 2, for example, glass, plastic, silicon, quartz, or the like, or may be formed from a different material.

In the present embodiment, the introduction path 3 can be selectively opened and closed. The introduction path 3 is provided with an opening/closing unit 31 that opens and closes the introduction path 3. Hereinafter, the opening/closing unit 31 is also referred to as the first opening/closing unit. The opening/closing unit 31 is, for example, a valve (e.g., an electrically-actuated valve, a solenoid valve, a flow control valve, and/or the like). As a result, by opening the opening/closing unit 31, the introduction path 3 can introduce the cell suspension into the storage unit 2. On the other hand, by closing the opening/closing unit 31, the introduction path 3 cannot introduce the cell suspension into the storage unit 2. In some examples, the opening/closing unit 31, or valve, may be selectively opened and/or closed based on a respective control signal received from the control unit 9.

The discharge path 4 is configured to discharge the cell suspension, which is after the voltage is applied, from the storage unit 2. In the present embodiment, the discharge path 4 is made with the tubular member. The discharge path 4 may be formed integrally with the storage unit 2 in advance or may be attached by the valve, the connector, or the like so that the liquid such as the cell suspension does not leak to the outside from the connection part with the storage unit 2. The discharge path 4 may be formed from the same material as the storage unit 2, for example, glass, plastic, silicon, quartz, or the like, or may be formed from a different material.

In the present embodiment, the discharge path 4 can be selectively opened and closed. The discharge path 4 is provided with an opening/closing unit 41 that opens and closes the discharge path 4. Hereinafter, the opening/closing unit 41 is also referred to as the second opening/closing unit. The opening/closing unit 41 is, for example, a valve (e.g., an electrically-actuated valve, a solenoid valve, a flow control valve, and/or the like). As a result, by opening the opening/closing unit 41, the discharge path 4 can discharge the cell suspension from the storage unit 2. On the other hand, by closing the opening/closing unit 41, the discharge path 4 cannot discharge the cell suspension from the storage unit 2. In some examples, the opening/closing unit 41, or valve, may be selectively opened and/or closed based on a respective control signal received from the control unit 9.

The filling unit 5 is configured to fill the predetermined amount of the cell suspension from the introduction path 3 into the storage unit 2. The predetermined amount is determined according to the amount of the cell suspension to be processed in one electroporation, and is, for example, 10 microliters to 10 milliliters, and desirably 40 microliters to 400 microliters. In the present embodiment, the filling unit 5 is, for example, a syringe (e.g., a tube and plunger pump, etc.). When the filling unit 5 is the syringe, in a state where the opening/closing unit 31 of the introduction path 3 is opened and the opening/closing unit 41 of the discharge path 4 is closed, the filling unit 5 can fill the predetermined amount of the cell suspension from the introduction path 3 into the storage unit 2 by pulling out a gasket 51, or plunger end, from the storage unit 2 side while maintaining airtightness of the storage unit 2. However, the filling unit 5 is not limited to the syringe, and may be an arbitrary instrument such as a suction pump capable of filling a stored object such as air from the storage unit 2. When the filling unit 5 is the suction pump, the filling unit 5 can fill the predetermined amount of the cell suspension from the introduction path 3 into the storage unit 2 by depressurizing the internal space of the storage unit 2. In any case, the filling unit 5 corresponds a pump that is capable of creating a positive or negative pressure inside the storage unit 2. The filling unit 5 may be an electrically-actuated pump that is capable of being selectively actuated based on a particular control signal received from the control unit 9.

The filling unit 5 may further discharge the predetermined amount of the cell suspension from the storage unit 2 to the discharge path 4. More specifically, the filling unit 5 can discharge the predetermined amount of the cell suspension from the storage unit 2 to the discharge path 4 by pushing the gasket 51 toward the storage unit 2 side in a state where the opening/closing unit 41 of the discharge path 4 is opened. When the filling unit 5 is the suction pump, the filling unit 5 can discharge the predetermined amount of the cell suspension from a substance introduction unit 22 to the discharge path 4 by pressurizing the internal space of the storage unit 2.

The electrode pair 6 is configured to apply the voltage to the cell suspension stored in the storage unit 2. The electrode pair 6 is attached to at least a part of the side surface of the storage unit 2 so as to sandwich the internal space of the storage unit 2. The electrode pair 6 is supplied with electric power from a power supply 61 in a state where the cell suspension is stored in the storage unit 2, thereby generating the electric pulse in the internal space of the storage unit 2 sandwiched between the electrode pair 6. As a result, the electroporation is performed in the storage unit 2, and the substance is introduced into the cells contained in the cell suspension. In some examples, the electrode pair may be caused to selectively apply voltage based on a control signal received from the control unit 9. In the storage unit 2, a part where the electrode pair 6 is provided on the side surface is also referred to as the substance introduction unit 22. The distance between the electrodes constituting the electrode pair 6 is a suitable distance for performing the electroporation, for example, 0.1 millimeters to 1 centimeter, but desirably a range from 1 millimeter to 5 millimeters. The electrode material is an electrically conductive substance, and may be, for example, a metal or a conductive polymer.

In the present embodiment, the electrode pair 6 is provided on the side surface of the storage unit 2 sandwiched between the introduction port 2A and the discharge port 2B in the longitudinal direction of the storage unit 2. That is, the storage unit 2 has the substance introduction unit 22 in a part sandwiched between the introduction port 2A and the discharge port 2B in the longitudinal direction. As a result, the substance introduction device 1A is installed such that the introduction port 2A is located higher than the discharge port 2B, whereby the cell suspension introduced from the introduction port 2A into the storage unit 2 is preferentially introduced by gravity to a part sandwiched between the electrode pair 6 of the storage unit 2 in the internal space of the storage unit 2. With such a configuration, the substance introduction device 1A can further improve an introduction rate of the substance into the cell in the electroporation. The substance introduction unit 22 may be formed from the same material as the other part of the storage unit 2, or may be formed from a different material. The substance introduction unit 22 may be formed from, for example, an insulating material such as glass, plastic, silicon, or quartz.

In the substance introduction device 1A, the storage unit 2 and the electrode pair 6 may be integrally provided. The storage unit 2 and the electrode pair 6 may be cuvette electrodes.

The resistance measurement unit 7 is configured to measure the resistance value between the electrode pair 6. The resistance measurement unit 7 includes, for example, an electrical sensor such as a resistance meter, a voltmeter, and/or an ammeter.

The liquid amount measurement unit 8 is configured to measure the liquid amount of the cell suspension stored in the storage unit 2. The liquid amount measurement unit 8 includes a sensor such as an infrared sensor, a weight sensor, and/or a level sensor.

The control unit 9 includes, for example, a memory 91 and a processor 92.

The memory 91 is, for example, a semiconductor memory, a magnetic memory, an optical memory, or the like. The memory 91 functions as, for example, a main storage device, an auxiliary storage device, or a cache memory. The memory 91 stores information used for the operation of the substance introduction device 1A. For example, the memory 91 stores a system program, an application program, embedded software, and/or the like.

The processor 92 may be, for example, a general-purpose processor such as a central processing unit (CPU), a dedicated processor specialized for specific processing, or the like. The processor 92 may include, for example, a dedicated circuit such as a field-programmable gate array (FPGA) or an application specific integrated circuit (ASIC).

As indicated by broken lines in FIG. 1, the control unit 9 is connected to each of the opening/closing unit 31 of the introduction path 3, the opening/closing unit 41 of the discharge path 4, the filling unit 5, the electrode pair 6, the resistance measurement unit 7, and the liquid amount measurement unit 8 so as to be able to communicate with the components by wire or wirelessly. As a result, the control unit 9 controls each unit of the substance introduction device 1A such as the opening/closing unit 31 of the introduction path 3, the opening/closing unit 41 of the discharge path 4, the filling unit 5, the electrode pair 6, the resistance measurement unit 7, and the liquid amount measurement unit 8. In some examples, the control unit 9 may send signals (e.g., control signals, etc.) to the opening/closing units 31, 41, the filling unit 5, the electrode pair 6, the resistance measurement unit 7, and/or the liquid amount measurement unit 8. These control signals may cause one or more of the opening/closing units 31, 41, the filling unit 5, the electrode pair 6, the resistance measurement unit 7, and the liquid amount measurement unit 8 to perform a respective function or functions associated with each device/component. Additionally or alternatively, the control unit 9 may receive signals from the opening/closing units 31, 41, the filling unit 5, the electrode pair 6, the resistance measurement unit 7, and/or the liquid amount measurement unit 8. The signals received by the control unit 9 may include data from one or more of the devices/components of the substance introduction device 1A. For example, the control unit 9 may receive signals from the resistance measurement unit 7 including resistance value data measured at one or more times. As another example, the control unit 9 may receive signals from the liquid amount measurement unit 8 including liquid level value data measured in the storage unit 2 at one or more times. Similar signals may be received from the other devices/components of the substance introduction device 1A.

The present embodiment describes as the substance introduction device 1A includes the control unit 9. However, the control unit 9 may be provided separately from the substance introduction device 1A. In such a case, the control unit 9 may be an information processing device of, for example, an electroporator, a personal computer, a smartphone, or a tablet terminal.

The operation of the substance introduction device 1A according to the present embodiment will be described with reference to FIG. 2. FIG. 2 is a flowchart illustrating the operation of the substance introduction device 1A according to the first embodiment. This operation corresponds to the substance introduction method executed by the substance introduction device 1A according to the present embodiment. In the present embodiment, the introduction path 3 of the substance introduction device 1A is described as being connected to the unprocessed bag 10 storing the cell suspension before the electroporation is performed in a state where the opening/closing unit 31 is closed. Furthermore, the discharge path 4 of the substance introduction device 1A is described as being connected to the processed bag 20 storing the cell suspension after the electroporation is performed in a state where the opening/closing unit 41 is closed.

At step S101: The control unit 9 controls the filling unit 5 to fill the storage unit 2 with the predetermined amount of the cell suspension.

The control unit 9 controls opening and closing of at least one of the opening/closing unit 31 of the introduction path 3 and the opening/closing unit 41 of the discharge path 4, and controls introduction of the cell suspension into the storage unit 2 or discharge of the cell suspension. Specifically, the control unit 9 controls the opening/closing unit 31 of the introduction path 3 so that the opening/closing unit 31 of the introduction path 3 is opened and the opening/closing unit 41 of the discharge path 4 is closed. The control unit 9 controls the filling unit 5 to fill the cell suspension from the unprocessed bag 10 to the storage unit 2 so that the liquid amount measured by the liquid amount measurement unit 8 falls within the predetermined range.

At step S102: The control unit 9 controls the resistance measurement unit 7 to determine whether the resistance value between the electrode pair 6 falls within the predetermined range.

Specifically, the control unit 9 controls the resistance measurement unit 7 in a state where the cell suspension is stored in the storage unit 2, and measures the resistance value between the electrode pair 6. The control unit 9 determines whether the resistance value between the electrode pair 6 falls within the predetermined range.

When the control unit 9 determines that the resistance value between the electrode pair 6 does not fall within the predetermined range (e.g., “No” at step S102), a process of step S103 is executed. On the other hand, when the control unit 9 determines that the resistance value between the electrode pair 6 falls within the predetermined range (e.g., “Yes” at step S102), a process of step S104 is executed.

At step S103: The control unit 9 controls the filling unit 5 to adjust the amount of the cell suspension stored in the storage unit 2 so that the resistance value measured by the resistance measurement unit 7 falls within the predetermined range.

Specifically, when the resistance value measured by the resistance measurement unit 7 falls below the predetermined range, the control unit 9 controls the filling unit 5 to fill the cell suspension from the unprocessed bag 10 into the storage unit 2. When the resistance value measured by the resistance measurement unit 7 exceeds the predetermined range, the control unit 9 controls the filling unit 5 to push the cell suspension back from the storage unit 2 to the unprocessed bag 10. Thereafter, the control unit 9 executes the process of step S102 again.

At step S104: The control unit 9 executes the electroporation.

Specifically, the control unit 9 controls the opening/closing unit 31 of the introduction path 3 to close both the opening/closing unit 31 of the introduction path 3 and the opening/closing unit 41 of the discharge path 4. The control unit 9 supplies electric power to the electrode pair 6 from the power supply 61 to apply a voltage to the cell suspension stored in the storage unit 2.

At step S105: The control unit 9 controls the filling unit 5 to discharge the cell suspension, which is after the voltage is applied, from the storage unit 2.

Specifically, the control unit 9 controls the opening/closing unit 41 of the discharge path 4 so that the opening/closing unit 31 of the introduction path 3 is closed and the opening/closing unit 41 of the discharge path 4 is opened. The control unit 9 controls the filling unit 5 to discharge the cell suspension, which is after the voltage is applied, from the storage unit 2 to the processed bag 20.

At step S106: The control unit 9 determines whether to end the present process, or method.

Specifically, the control unit 9 determines whether the present process has been repeated a predetermined number of times. When the control unit 9 determines that the present process has not repeated the predetermined number of times (e.g., “No” at S106), the process of step S101 is executed again. On the other hand, the control unit 9 determines that the present process has repeated the predetermined number of times (e.g., “Yes” at step S106), the present process is ended. The control unit 9 may determine whether the total amount of the cell suspension subjected to the present process has reached the predetermined amount instead of determining whether the present process has repeated the predetermined number of times. Once the process is ended, the processed bag 20 may be detached from the discharge path 4 and/or the storage unit 2.

In the above-described processing, the control unit 9 is described as controlling the opening/closing unit 31, the opening/closing unit 41, and the filling unit 5 for filling the cell suspension into the storage unit 2 and discharging the cell suspension. However, the control unit 9 may control the filling of the cell suspension into the storage unit 2 and discharging of the cell suspension without controlling the filling unit 5 or by partially controlling the filling unit 5.

For example, by using the fact that the cell suspension in the unprocessed bag 10 is pushed out by its own weight when the unprocessed bag 10 connected to the introduction path 3 is arranged at a position higher than the substance introduction device 1, the control unit 9 may fill the storage unit 2 with the predetermined amount of cell suspension only by opening and closing the opening/closing unit 31. Alternatively, the unprocessed bag 10 may be connected to the introduction path 3 in a state where the inside of the unprocessed bag 10 is maintained at a positive pressure. By using the fact that the cell suspension is pushed out from the unprocessed bag 10, the control unit 9 can fill the storage unit 2 with the predetermined amount of the cell suspension by closing the opening/closing unit 31 when the cell suspension stored in the storage unit 2 reaches the predetermined amount after the opening/closing unit 31 is opened. Similarly, the control unit 9 may discharge the predetermined amount of the cell suspension from the storage unit 2 only by opening and closing the opening/closing unit 41 by using the fact that the cell suspension in the storage unit 2 is pushed out by its own weight.

Furthermore, in a case where the filling or discharging of the cell suspension cannot be sufficiently performed only by opening and closing the opening/closing unit 31 or the opening/closing unit 41, the control unit 9 may control the filling unit 5 to control the filling or discharging of the predetermined amount of the cell suspension. With such a configuration, the substance introduction device 1A can reduce power required for controlling the filling unit 5 and achieve energy saving.

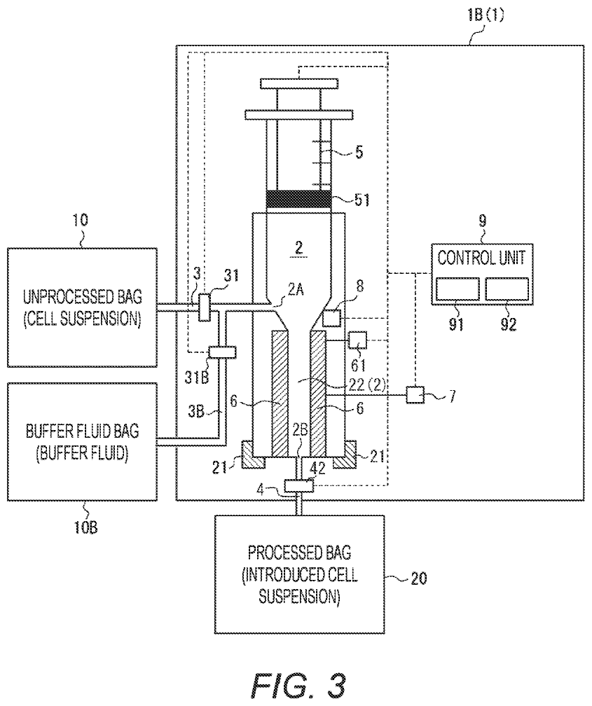

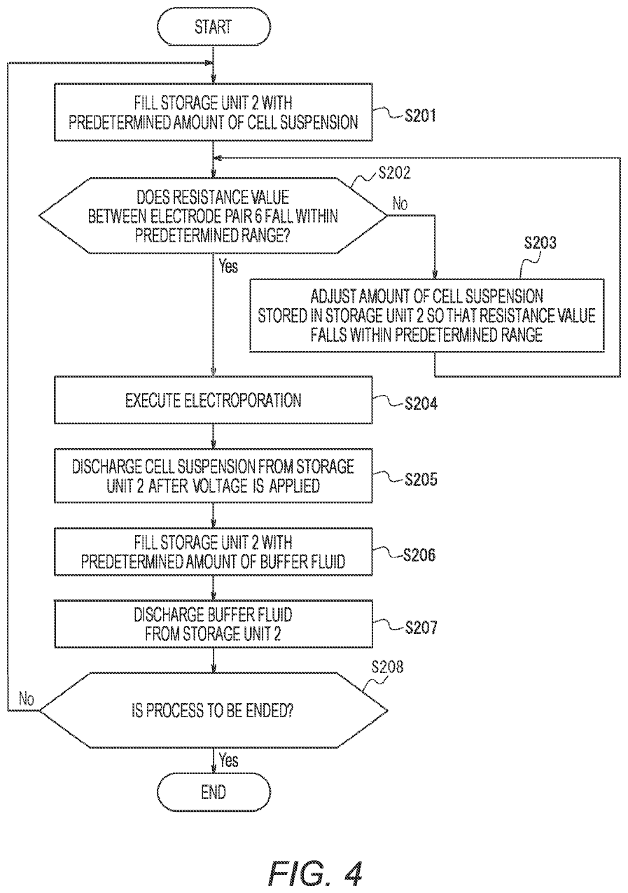

A substance introduction device 1B, which is a second embodiment of the substance introduction device 1 according to the present disclosure, will be described with reference to FIGS. 3 and 4. FIG. 3 is a schematic diagram illustrating a schematic configuration of the substance introduction device 1B according to the second embodiment. FIG. 4 is a flowchart illustrating the operation of the substance introduction device 1B according to the second embodiment.

The second embodiment is different from the first embodiment in that the substance introduction device 1B includes an auxiliary introduction path 3B. Hereinafter, the second embodiment will be described focusing on differences from the first embodiment. Note that parts having the same configurations as those of the first embodiment are denoted by the same reference numerals.

As illustrated in FIG. 3, the substance introduction device 1B includes the auxiliary introduction path 3B in addition to the storage unit 2, the introduction path 3, the discharge path 4, the filling unit 5, the electrode pair 6, the resistance measurement unit 7, the liquid amount measurement unit 8, and the control unit 9.

The auxiliary introduction path 3B is configured to introduce a liquid different from the cell suspension into the storage unit 2. The liquid different from the cell suspension is, for example, the buffer fluid. In the present embodiment, the auxiliary introduction path 3B is made with the tubular member. The auxiliary introduction path 3B communicates with the introduction path 3 outside the introduction port 2A of the storage unit 2 so as to communicate with the introduction port 2A of the storage unit 2. The introduction path 3 may be formed integrally with the auxiliary introduction path 3B in advance or may be attached by the valve, the connector, or the like so that a liquid does not leak to the outside from a connection part with the introduction path 3 and the auxiliary introduction path 3B. The auxiliary introduction path 3B may be formed from the same material as the introduction path 3, for example, glass, plastic, silicon, quartz, or the like, or may be formed from a different material.

In the present embodiment, the auxiliary introduction path 3B can be opened and closed. The auxiliary introduction path 3B is provided with an opening/closing unit 31B that opens and closes the auxiliary introduction path 3B. The opening/closing unit 31B is, for example, a valve (e.g., an electrically-actuated valve, a solenoid valve, a flow control valve, and/or the like). As a result, by opening the opening/closing unit 31B, the auxiliary introduction path 3B can introduce the buffer fluid into the storage unit 2. As a result, by closing the opening/closing unit 31B, the auxiliary introduction path 3B cannot introduce the buffer fluid into the storage unit 2. The opening/closing unit 31B is connected to the control unit 9 so as to be able to communicate with the control unit 9 by wire or wirelessly. In some examples, the opening/closing unit 31B, or valve, in a similar manner as the opening/closing unit 31 described above. For instance, the opening/closing unit 31B may be selectively opened and/or closed based on a respective control signal received from the control unit 9.

This operation for describing the operation of the substance introduction device 1B according to the present embodiment with reference to FIG. 4 corresponds to the substance introduction method executed by the substance introduction device 1B according to the present embodiment. In the present embodiment, the introduction path 3 is connected to the unprocessed bag 10 storing the cell suspension before the electroporation is performed in a state where the opening/closing unit 31 is closed. The auxiliary introduction path 3B is connected to a buffer fluid bag 10B storing the buffer fluid in a state where the opening/closing unit 31B is closed. Furthermore, the discharge path 4 is connected to the processed bag 20 storing the cell suspension after the electroporation is performed in a state where the opening/closing unit 41 is closed.

At step S201: As described above at step S101, the control unit 9 controls the filling unit 5 to fill the storage unit 2 with the predetermined amount of the cell suspension.

At step S202: As described above at step S102, the control unit 9 controls the resistance measurement unit 7 to determine whether the resistance value between the electrode pair 6 falls within the predetermined range.

When the control unit 9 determines that the resistance value between the electrode pair 6 does not fall within the predetermined range (e.g., “No” at step S202), a process of step S203 is executed. On the other hand, when the control unit 9 determines that the resistance value between the electrode pair 6 falls within the predetermined range (e.g., “Yes” at step S202), a process of step S204 is executed.

At step S203: As described above at step S103, the control unit 9 controls the filling unit 5 to adjust the amount of the cell suspension stored in the storage unit 2 so that the resistance value measured by the resistance measurement unit 7 falls within the predetermined range. Thereafter, the control unit 9 executes the process of step S202 again.

At step S204: As described above at step S104, the control unit 9 executes the electroporation.

At step S205: As described above at step S105, the control unit 9 controls the filling unit 5 to discharge the cell suspension which is after the voltage is applied from the storage unit 2.

At step S206: The control unit 9 controls the filling unit 5 to fill the storage unit 2 with the predetermined amount of the buffer fluid from the auxiliary introduction path 3B.

Specifically, the control unit 9 controls the opening/closing unit 31B of the auxiliary introduction path 3B and the opening/closing unit 41 of the discharge path 4 so that the opening/closing unit 31B of the auxiliary introduction path 3B is opened and both the opening/closing unit 31 of introduction path 3 and the opening/closing unit 41 of the discharge path 4 are closed. The control unit 9 controls the filling unit 5 to fill the storage unit 2 with the buffer fluid from the buffer fluid bag 10B. In the present embodiment, the amount of the buffer fluid filled in the storage unit 2 in step S206 is the same as the amount of the cell suspension filled in the storage unit 2 in step S201, but it may be different.

At step S207: The control unit 9 controls the filling unit 5 to discharge the buffer fluid from the storage unit 2.

Specifically, the control unit 9 controls the auxiliary introduction path 3B of the opening/closing unit 31B and the opening/closing unit 41 of the discharge path 4 so that the opening/closing unit 31 of the introduction path 3 and the opening/closing unit 31B of the auxiliary introduction path 3B are closed and the opening/closing unit 41 of the discharge path 4 is opened. The control unit 9 controls the filling unit 5 to discharge the buffer fluid from the storage unit 2 to the processed bag 20.

At step S208: As described above at step S106, the control unit 9 determines whether to end the present process, or method.

When the control unit 9 determines that the present process has not repeated the predetermined number of times (e.g., “No” at S208), the process of step S201 is executed again. On the other hand, the control unit 9 determines that the present process has repeated the predetermined number of times (e.g., “Yes” at step S208), the present process is ended.

Additionally or alternatively, the substance introduction device 1B can alternate between performing the electroporation on the cell suspension received from the unprocessed bag 10 and washing of the storage unit 2 with the buffer fluid received from the buffer fluid bag 10B. In some examples, the process may include a step of removing the processed bag 20 from the substance introduction device 1B after the electroporation is performed and the cell suspension is discharged into the processed bag 20 (e.g., after step S205). In one example, the process may include washing of the storage unit 2, by continuing at step S206 (e.g., after the processed bag 20 is removed) and discharging the buffer fluid from the storage unit 2 (e.g., via the discharge path 4, etc.), at step S207. In this example, rather than discharging the buffer fluid to the processed bag 20, which has been removed, the buffer fluid may be flushed through the storage unit and discharged via the discharge path 4 (e.g., to a container) thereby cleaning the storage unit 2.

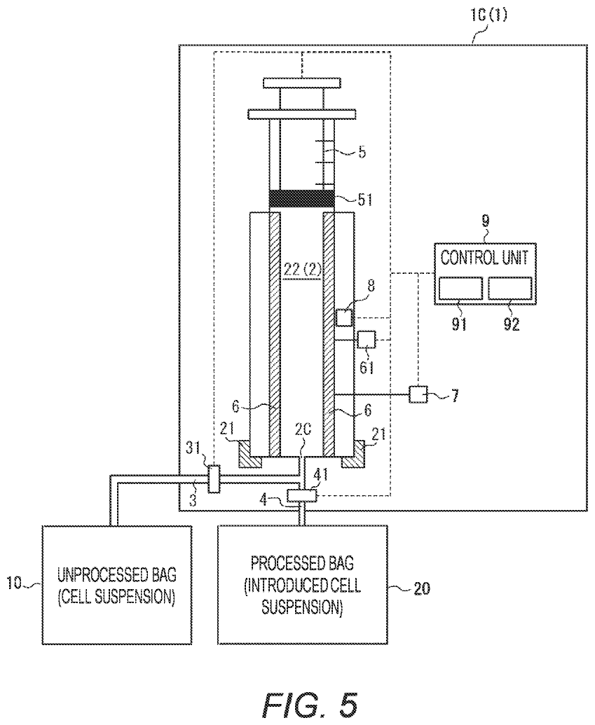

A substance introduction device 1C, which is a third embodiment of the substance introduction device 1 according to the present disclosure, will be described with reference to FIG. 5. FIG. 5 is a schematic diagram illustrating a schematic configuration of the substance introduction device 1C according to the third embodiment.

The third embodiment is different from the first embodiment in that one opening 2C serving as both the introduction port 2A and the discharge port 2B is provided in the storage unit 2 of the substance introduction device 1C, and both the introduction path 3 and the discharge path 4 communicate with the opening 2C. In the present embodiment, the opening 2C is provided at one end different from the one end to which the filling unit 5 is connected in the longitudinal direction of the storage unit 2. The substance introduction device 1C of the present embodiment does not include the auxiliary introduction path 3B indicated in the second embodiment described above, but may include the auxiliary introduction path 3B. The auxiliary introduction path 3B may communicate with, for example, the introduction path 3 or the discharge path 4, or may communicate with the internal space of the storage unit 2 separately from the introduction path 3 and the discharge path 4.

Further, in the substance introduction device 1C, the electrode pair 6 is provided on the side surface of the storage unit 2 over the longitudinal direction of the storage unit 2. In such a case, the entire storage unit 2 is the substance introduction unit 22. With such a configuration, the substance introduction device 1C can further improve the introduction rate of the substance into the cell in the electroporation. The substance introduction devices 1A and 1B described above in the first embodiment and the second embodiment do not include the electrode pair 6 in the longitudinal direction of the storage unit 2 as in the present embodiment, but may include the electrode pair 6 in the longitudinal direction of the entire storage unit 2.

As described above, the substance introduction device 1 according to the present disclosure is the substance introduction device 1 that introduces the substance into the cell by the electroporation, the substance introduction device 1 including: the storage unit 2 that stores the cell suspension containing the cells and the substance; the introduction path 3 that introduces the cell suspension into the storage unit 2; the filling unit 5 that fills the storage unit 2 with the predetermined amount of the cell suspension from the introduction path 3; the electrode pair 6 that applies a voltage to the cell suspension stored in the storage unit 2; and the discharge path 4 that discharges the cell suspension after the voltage is applied from the storage unit 2. With such a configuration, since the substance introduction device 1 can repeatedly perform the electroporation for each predetermined amount of cell suspension, the electroporation can be repeated under the same conditions for performing even when the total amount of the cell suspension to be electroporated is changed. Therefore, the substance introduction device 1 can improve the usefulness of the technique for introducing the substance into cells by electroporation.

The substance introduction device 1 as one embodiment can further include the control unit 9 that controls the filling unit 5 to fill the predetermined amount of the cell suspension in the storage unit 2. With such a configuration, the substance introduction device 1 can automatically execute the electroporation.

The substance introduction device 1 as one embodiment can further include the opening/closing unit 31 that opens and closes the introduction path 3 and an opening/closing unit 41 that opens and closes the discharge path 4, and the control unit 9 can control opening and closing of at least one of the opening/closing unit 31 and the opening/closing unit 41, and controls introduction of the cell suspension into the storage unit 2 or discharge of the cell suspension. With such a configuration, the substance introduction device 1 can reduce the risk of introducing the cell suspension into the storage unit 2 or discharging the cell suspension regardless of the control of the filling unit 5, and more accurately control the amount of the cell suspension to be filled in the storage unit 2.

The substance introduction device 1 as one embodiment can further include the liquid amount measurement unit 8 that measures the liquid amount of the cell suspension stored in the storage unit 2, and the control unit 9 can control the filling unit 5 to fill the storage unit 2 with the cell suspension so that the liquid amount measured by the liquid amount measurement unit 8 falls within the predetermined range. With such a configuration, the substance introduction device 1 can further improve the introduction rate of the substance into cells in the electroporation by more accurately measuring the cell suspension to be subjected to one electroporation.

The substance introduction device 1 as one embodiment can further include the resistance measurement unit 7 that measures the resistance value between the electrode pair 6, and the control unit 9 can control the filling unit 5 to adjust the amount of the cell suspension stored in the storage unit 2 so that the resistance value measured by the resistance measurement unit 7 falls within the predetermined range. With such a configuration, the substance introduction device 1 can further improve the introduction rate of the substance into cells in the electroporation by adjusting the amount of the cell suspension to be subjected to one electroporation based on the resistance value.

The substance introduction device 1 as one embodiment can further include the auxiliary introduction path 3B that introduces a liquid different from the cell suspension into the storage unit 2, and the filling unit 5 can fill the storage unit 2 with the predetermined amount of liquid from the auxiliary introduction path 3B. With such a configuration, the substance introduction device 1 can perform the electroporation on the cell suspension and washing of the storage unit 2 with a liquid such as the buffer fluid. As a result, the substance introduction device 1 can reduce the probability that the cell remains in the storage unit 2 after the voltage is applied. Therefore, the substance introduction device 1 can further improve the introduction rate of the substance into cells in the electroporation and reduce the number of unrecovered substance-introduced cells.

In the substance introduction device 1 as one embodiment, the introduction port 2A through which the introduction path 3 communicates with the storage unit 2 may be provided at a position different from a position of the discharge port 2B through which the discharge path 4 communicates with the storage unit 2. With such a configuration, the substance introduction device 1 can reduce the probability that the cell suspension after the electroporation is mixed into an unprocessed cell suspension before the electroporation is performed.

In the substance introduction device 1 as one embodiment, the storage unit 2 is detachable from the substance introduction device 1. With such a configuration, the substance introduction device 1 can perform the electroporation by replacing the storage unit 2 when the storage unit 2 fails, the upper limit of the number of uses is reached, or the like.

The substance introduction method executed by the substance introduction device 1 according to the present disclosure includes filling the storage unit 2 with the predetermined amount of the cell suspension, and applying a voltage to the cell suspension stored in the storage unit 2. With such a configuration, since the substance introduction method can repeatedly perform the electroporation for each predetermined amount of cell suspension, the electroporation can be repeated under the same conditions for performing even when the total amount of the cell suspension to be electroporated is changed. Therefore, the substance introduction method can improve the usefulness of the technique for introducing the substance into cells by electroporation.

The substance introduction device 1 according to the present disclosure is the substance introduction device 1 that introduces the substance into cells by the electroporation, the substance introduction device 1 including: the storage unit 2 that stores the cell suspension containing the cells and the substance; the introduction path 3 that introduces the cell suspension into the storage unit 2; the electrode pair 6 that applies a voltage to the cell suspension stored in the storage unit 2; the discharge path 4 that discharges the cell suspension after the voltage is applied from the storage unit 2; the opening/closing unit 31 that opens and closes the introduction path 3; the opening/closing unit 41 that opens and closes the discharge path 4; and the control unit 9 that controls opening and closing of at least one of the opening/closing unit 31 and the opening/closing unit 41, and controls introduction of the cell suspension into the storage unit 2 or discharge of the cell suspension. With such a configuration, the substance introduction device 1 can repeatedly perform electroporation for every predetermined amount of cell suspension without controlling the filling unit 5 or by partially controlling the filling unit 5, for example, by using the fact that the cell suspension is pushed out from the unprocessed bag 10 connected to the introduction path 3. Therefore, the substance introduction device 1 can improve the usefulness of the technique for introducing the substance into cells by electroporation.

Although the present disclosure has been described with reference to the drawings and examples, it should be noted that those skilled in the art can make various modifications and corrections based on the present disclosure. Accordingly, it should be noted that these variations and modifications fall within the scope of the present disclosure. For example, functions or the like included in each means, each step, or the like can be rearranged so as not to be logically inconsistent, and a plurality of means, steps, or the like can be combined into one or divided.

For example, in the above-described embodiments, it has been described that the number of the storage unit 2 included in the substance introduction device 1 is one. However, the substance introduction device 1 may include a plurality of storage units 2. In such a case, the substance introduction device 1 may include a plurality of introduction paths 3, discharge paths 4, filling units 5, electrode pairs 6, resistance measurement units 7, and liquid amount measurement units 8 according to the number of the storage units 2. The control unit 9 of the substance introduction device 1 may uniformly control the plurality of filling units 5 to fill the predetermined amount of cell suspension in each of the plurality of storage units 2 simultaneously, or may individually control each of the plurality of filling units 5 to fill the predetermined amount of cell suspension in each of the plurality of storage units 2 at different times.

Alternatively, in the above-described embodiments, the substance introduction device 1 has been described as including the liquid amount measurement unit 8. However, the substance introduction device 1 does not necessarily include the liquid amount measurement unit 8. In such a case, the control unit 9 of the substance introduction device 1 may control the filling unit 5 by storing a control amount or a control time of the filling unit 5 for filling the predetermined amount of the cell suspension from the introduction path 3 into the storage unit 2 in the memory 91 in advance. For example, the liquid amount of the cell suspension to be filled in the storage unit 2 at one time may be controlled based on a movement amount of a plunger of the syringe as the filling unit 5. As a result, the substance introduction device 1 can be manufactured by a simpler manufacturing method, and a manufacturing cost can be reduced.

Furthermore, in the above-described embodiments, the liquid amount of the cell suspension filled in the storage unit 2 is controlled by the control unit 9. However, the liquid amount of the cell suspension to be filled in the storage unit 2 may be manually adjusted by, for example, an operation of the syringe as the filling unit 5 and visual observation by an operator.

What is claimed is:

1. A substance introduction device that introduces a substance into at least one cell by electroporation, the substance introduction device comprising:
a storage unit that stores a cell suspension containing the at least one cell and the substance;
an introduction path that introduces the cell suspension into the storage unit;
a filling unit that fills the storage unit with a predetermined amount of the cell suspension from the introduction path;
an electrode pair that applies a voltage to the cell suspension stored in the storage unit; and
a discharge path that discharges the cell suspension from the storage unit after the voltage is applied.

2. The substance introduction device of claim 1, further comprising a control unit that controls the filling unit to fill the predetermined amount of the cell suspension in the storage unit.

3. The substance introduction device of claim 2, further comprising:
a first opening/closing unit that selectively opens and closes the introduction path; and
a second opening/closing unit that selectively opens and closes the discharge path,
wherein the control unit controls opening and closing of at least one of the first opening/closing unit and the second opening/closing unit, and controls at least one of introduction of the cell suspension into the storage unit and discharge of the cell suspension.

4. The substance introduction device of claim 2, further comprising:
a liquid amount measurement unit that measures a liquid amount of the cell suspension stored in the storage unit,
wherein the control unit controls the filling unit to fill the storage unit with the cell suspension so that the liquid amount measured by the liquid amount measurement unit falls within a predetermined range.

5. The substance introduction device of claim 2, further comprising:
a resistance measurement unit that measures a resistance value between the electrode pair,
wherein the control unit controls the filling unit to adjust the predetermined amount of the cell suspension stored in the storage unit so that the resistance value measured by the resistance measurement unit falls within a predetermined range.

6. The substance introduction device of claim 1, further comprising:
an auxiliary introduction path that introduces a liquid different from the cell suspension into the storage unit,
wherein the filling unit fills the storage unit with a predetermined amount of the liquid from the auxiliary introduction path.

7. The substance introduction device of claim 1, further comprising:
an introduction port disposed at a first location of the storage unit, wherein the cell suspension is introduced along the introduction path and through the introduction port into the storage unit; and
a discharge port disposed at a second location of the storage unit, wherein the second location is different from the first location, and wherein the cell suspension is discharged along the discharge path and through the discharge port.

8. The substance introduction device of claim 1, wherein the storage unit is detachable from the substance introduction device.

9. A substance introduction method executed by a substance introduction device that introduces a substance into at least one cell by electroporation, the method comprising:
providing the substance introduction device, wherein the substance introduction device comprises:
a storage unit that stores a cell suspension containing the at least one cell and the substance;
an introduction path that introduces the cell suspension into the storage unit;
a filling unit that fills the storage unit with a predetermined amount of the cell suspension from the introduction path;
an electrode pair that applies a voltage to the cell suspension stored in the storage unit; and
a discharge path that discharges the cell suspension from the storage unit after the voltage is applied;.
filling the storage unit with the predetermined amount of the cell suspension; and
applying the voltage to the cell suspension stored in the storage unit.

10. The method of claim 9, wherein a control unit causes the filling unit to fill the predetermined amount of the cell suspension in the storage unit.

11. The method of claim 10, further comprising:
opening, by a first opening/closing unit, the introduction path when filling the storage unit with the predetermined amount of the cell suspension; and
wherein the control unit controls the opening and the closing of the first opening/closing unit.

12. The method of claim 10, further comprising:
measuring, by a liquid sensor, a liquid amount of the cell suspension stored in the storage unit; and
filling, by the filling unit, the storage unit with the cell suspension so that the liquid amount measured by the liquid sensor falls within a predetermined range.

13. The method of claim 10, further comprising:
measuring, by an electrical sensor, a resistance value between the electrode pair; and
adjusting, by the filling unit, an amount of the cell suspension stored in the storage unit so that the resistance value measured by the electrical sensor falls within a predetermined range.

14. The method of claim 9, further comprising:
filling, from an auxiliary introduction path, the storage unit with a predetermined amount of liquid.

15. The method of claim 9, further comprising:
connecting, prior to discharging the cell suspension from the storage unit after the voltage is applied, a processed bag to the discharge path.

16. A substance introduction device that introduces a substance into a cell by electroporation, the substance introduction device comprising:
a storage unit that stores a cell suspension containing the cell and the substance;
an introduction path that introduces the cell suspension into the storage unit;
an electrode pair that applies a voltage to the cell suspension stored in the storage unit;
a discharge path that discharges the cell suspension from the storage unit after the voltage is applied;
a first opening/closing unit that opens and closes the introduction path;
a second opening/closing unit that opens and closes the discharge path; and
a control unit that controls opening and closing of at least one of the first opening/closing unit and the second opening/closing unit, and controls introduction of the cell suspension into the storage unit or discharge of the cell suspension.

17. The substance introduction device of claim 16, further comprising:
a liquid sensor that measures a liquid amount of the cell suspension stored in the storage unit,
wherein the control unit causes a filling unit to fill the storage unit with the cell suspension such that the liquid amount measured by the liquid sensor falls within a predetermined range.

18. The substance introduction device of claim 17, further comprising:
an electrical sensor that measures a resistance value between the electrode pair,
wherein the control unit controls the filling unit to adjust an amount of cell suspension stored in the storage unit such that the resistance value measured by the electrical sensor falls within a predetermined range.

19. The substance introduction device of claim 16, further comprising:
an auxiliary introduction path that introduces a liquid different from the cell suspension into the storage unit,
wherein a pump fills the storage unit with a predetermined amount of the liquid from the auxiliary introduction path.

20. The substance introduction device of claim 16, wherein the storage unit is detachable from the substance introduction device.
